(19) United States
(12) Patent Application Publication
(10) Pub. No.: US 2019/0167230 A1
(43) Pub. Date: Jun. 6, 2019

(54) ULTRASONIC PROBE

(71) Applicant: SAMSUNG MEDISON CO., LTD., Hongcheon-gun (KR)

(72) Inventors: Young Mun CHO, Seoul (KR); Kyung Hun SONG, Seongnam-si (KR)

(21) Appl. No.: 15/868,884

(22) Filed: Jan. 11, 2018

(30) Foreign Application Priority Data
Dec. 1, 2017 (KR) 10-2017-0164041

(51) Int. Cl. A61B 8/00; A61L 2/10

(52) U.S. Cl. CPC A61B 8/4422; A61B 8/4405; A61L 2202/24; A61L 2/10; A61B 8/4444

(57) ABSTRACT

There is disclosed an ultrasonic probe, and more particularly, an ultrasonic probe configured to visually provide a change based on an ultraviolet (UV) response during UV disinfection. The ultrasonic probe according to one aspect of the present disclosure is provided such that a coated layer formed of a UV-sensitive material is provided on a surface of the ultrasonic probe, and the UV-sensitive material provided in the coated layer is discolored.

### CROSS-REFERENCE TO RELATED APPLICATION

This application claims the benefit of Korean Patent Application No. 10-2017-0164041, filed on Dec. 1, 2017 in the Korean Intellectual Property Office, the disclosure of which is incorporated herein by reference.

### BACKGROUND

### 1. Field

Embodiments of the present disclosure relate to an ultrasonic probe, and more particularly, to an ultrasonic probe configured to visually provide a change based on an ultraviolet (UV) response during UV disinfection.

### 2. Description of the Related Art

Ultrasonic diagnostic apparatuses are apparatuses configured to irradiate a specific part of an object with an ultrasonic signal, receive a reflected ultrasonic signal (an ultrasonic echo signal) from the object, and acquire a tomographic image or an image of a blood flow of soft tissues using information received from the object in a non-invasive manner.

The ultrasonic diagnostic apparatuses have advantages of being relatively small and inexpensive in comparison to other image diagnostic apparatuses such as X-ray diagnostic apparatuses, X-ray computerized tomography (CT) scanners, and magnetic resonance imagings (MRIs). In addition, the ultrasonic diagnostic apparatus can acquire an image related to an inside of an object in real time, and have high stability because there is no exposure to radiation. Accordingly, the ultrasound diagnostic apparatuses are commonly used in diagnoses related to a human heart and abdomen as well as in urology and obstetrics and gynecology.

The ultrasonic diagnostic apparatus includes an ultrasonic probe for transmitting an ultrasonic signal to an object to acquire an ultrasonic image of an inside of the object and for receiving a response signal reflected from the object. Such an ultrasonic probe needs to be sterilized periodically to prevent infection caused by bacteria when used on a plurality of patients.

### SUMMARY

Therefore, it is an aspect of the present disclosure to provide an ultrasonic probe configured such that a coated layer formed of an ultraviolet (UV)-sensitive material is applied on an ultrasonic probe to provide a visualized change in the coated layer according to a UV response during UV disinfection.

Additional aspects of the disclosure will be set forth in part in the description which follows and, in part, will be obvious from the description, or may be learned by practice of the disclosure.

In accordance with one aspect of the present disclosure, an ultrasonic probe includes a coated layer formed on a surface thereof and formed of a UV-sensitive material having a property of light discoloration, wherein the UV-sensitive material provided in the coated layer is discolored during UV irradiation.

The coated layer may be coated with a pigment in which a powder type material having the property of light discoloration and at least one of an organic solvent and a water-soluble solution are mixed.

The coated layer may be formed on at least a part of a surface of the ultrasonic probe.

The ultrasonic probe may further include a case configured to accommodate a transducer module; and a lens installed at one end of the case, wherein the coated layer may be formed on at least one of the case and the lens.

The ultrasonic probe may further include a strain reliever provided at the other end of the case, wherein the coated layer may be formed on the strain reliever.

The UV-sensitive material having the property of light discoloration may include a UV-sensitive material having the property of light discoloration including at least one selected from a group consisting of a fulgide-based molecule, a diarylethene-based molecule, an azobenzene-based molecule, a spirooxazine-based molecule, and a spiropyran-based molecule.

The diarylethene-based molecule may include at least one selected from the group consisting of thiophene perfluoropentane, benzothiophene perfluoropentane, benzothiophene maleicanhydride, benzothiophene cyanoethene, a benzothiophene perfluoropentane derivative, a benzothiophene sulfone perfluoropetene derivative, a benzothiophene perfluoropetene polymer, and a benzothiophene sulfone perfluoropetene polymer.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a view illustrating an ultrasonic diagnostic apparatus according to one embodiment of the present disclosure;

FIG. 2 is a view illustrating an ultrasonic probe according to one embodiment of the present disclosure;

FIGS. 3 and 4 are views for describing a principle of light discoloration; and

FIG. 5 is a view illustrating a discoloration process of the ultrasonic probe according to one embodiment of the present disclosure.

### DETAILED DESCRIPTION

Embodiments described in the specification and structures illustrated in the drawings are exemplary embodiments of the disclosed disclosure and may cover various modifications that may be substituted for the embodiments herein and drawings at the time of filing of this application.

In addition, terms used in the present specification are merely used to describe exemplary embodiments and are not intended to limit and/or restrict the disclosed disclosure. An expression used in the singular encompasses the expression of the plural, unless it has a clearly different meaning in the context.

In the present specification, the terms such as “including,” “having,” and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

In addition, although the terms first, second, and the like may be used herein in reference to elements of the present disclosure, such elements are not to be construed as limited by the terms.

Hereinafter, embodiments of the present disclosure that are easily performed by those skilled in the art will be described in detail with reference to the accompanying drawings.

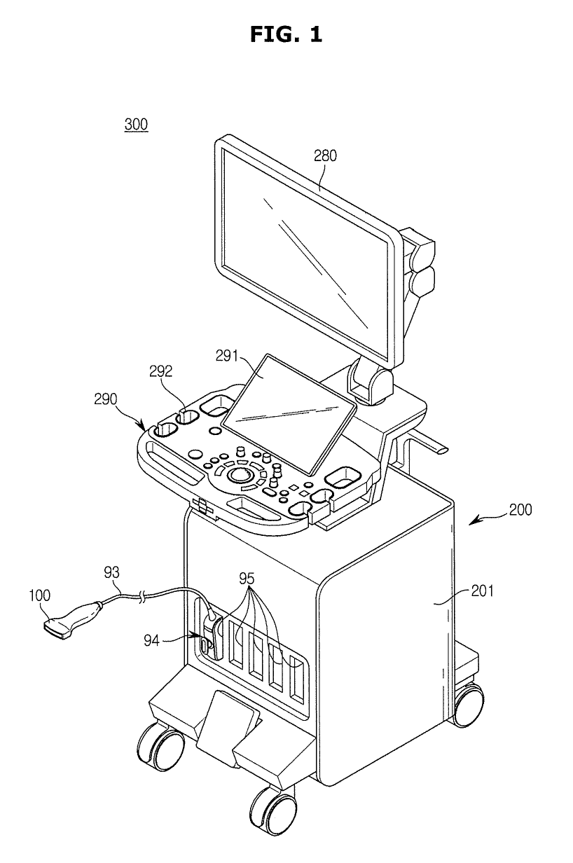

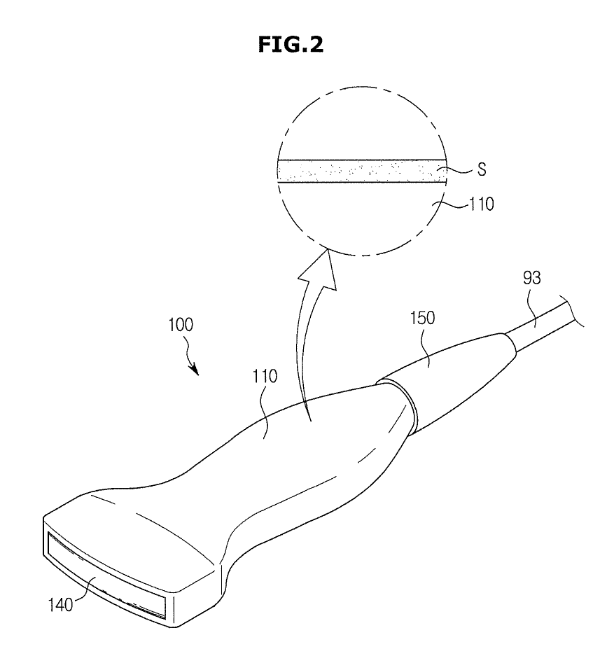

FIG. 1 is a perspective view illustrating an ultrasonic diagnostic apparatus 300 including an ultrasonic probe 100 according to one embodiment of, and FIG. 2 is a view illustrating an exterior of the ultrasonic probes 100 selected from various kinds of ultrasonic probes classified according to a shape of a transducer of the ultrasonic probe.

Referring to FIG. 1, the ultrasonic diagnostic apparatus 300 may include a main body 200, an input unit 290 through which a user inputs a command for controlling the ultrasonic diagnostic apparatus 300, and a display 280 configured to output information received from the input unit 290 and the main body 200.

Specifically, the main body 200 may control overall operation of the ultrasonic diagnostic apparatus 300, various components for controlling overall operations of the ultrasonic probe 100 and the main body 200 may be provided in the main body 200, and the main body 200 and the ultrasonic probe 100 may transmit and receive data using a connecting cable 93 or a wireless communication module.

In addition, as illustrated in FIG. 1, the ultrasonic probe 100 and the main body 200 may be connected using the connecting cable 93 to communicate. An electric signal output from the ultrasonic probe 100 via the connecting cable 93 may be transmitted to the main body 200. In addition, a control command and the like generated by the main body 200 may also be transmitted to the ultrasonic probe 100 via the connecting cable 93.

A connector 94 may be provided at one end of the connecting cable 93, and the connector 94 may be coupled to and separated from a port 95 provided in an exterior 201 of the main body 200. In a case in which the connector 94 is coupled to the port 95, the ultrasonic probe 100 and the main body 200 may be connected to be able to communicate with each other.

In addition, probe holders 292 on which the ultrasonic probe 100 may be placed may be provided in one side surface of the main body 200. As many probe holders 292 as the number of ultrasonic probes 100 may be provided and installed on or separated from the main body 200. In a case in which the user does not use the ultrasonic probe 100, the ultrasonic probe 100 may be placed and kept on the probe holder 293.

In addition, the main body 200 may receive an electric signal, which is output from the ultrasonic probe 100, from the ultrasonic probe 100 and transmit an electric signal generated by the main body 200 to the ultrasonic probe 100 via a wireless communication network. In this case, a wireless communication module including an antenna and a wireless communication chip may be installed in each of the ultrasonic probe 100 and the main body 200.

The wireless communication module may be a short-range wireless communication module using at least one among Bluetooth communication, Bluetooth low energy communication, infrared data association (IrDA) communication, Wi-Fi communication, Wi-Fi direct communication, Ultra-Wideband (UWB) communication, and near field communication (NFC), or a wireless communication module configured to support a Third Generation Partnership Project (3GPP) series or a Third Generation Partnership Project 2 (3GPP2) series certified by the International Telecommunication Union (ITU), or an Institute of Electrical and Electronics engineers (IEEE) series wireless communication network.

The main body 200 may transmit and receive data to and from servers or other medical apparatuses in a hospital connected via a picture archiving and communication system (PACS) using a communication portion. In addition, the main body 200 may transmit and receive data on the basis of standards of digital imaging and communications in medicine (DICOM), but is not limited thereto.

The display 280 may be coupled to the main body 200 and display various pieces of information received from the main body 200.

Specifically, the display 280 may display an ultrasonic image of a target part in an object. The ultrasonic image displayed on the display 280 may be a two dimensional ultrasonic image, a three dimensional ultrasonic image, or a Doppler image, and may be variously displayed according to an operation mode of the ultrasonic diagnostic apparatus 300.

According to one embodiment, the ultrasonic image includes an amplitude mode (A-mode) image, a brightness mode (B-mode) image, a motion mode (M-mode) image, a color-mode (C-mode) image, and a Doppler-mode (D-mode) image.

Hereinafter, the A-mode image denotes an ultrasonic image configured to show a magnitude of an ultrasonic signal corresponding to an echo ultrasonic signal, the B-mode image denotes an ultrasonic image configured to show a brightness as a magnitude of an ultrasonic signal corresponding to the echo ultrasonic signal, and the M-mode image denotes an ultrasonic image configured to show movement of an object at a specific position over time. The D-mode image denotes an ultrasonic image configured to show a waveform as a moving object using the Doppler effect, and the C-mode image denotes an ultrasonic image configured to show a color spectrum as a moving object.

Accordingly, the display 280 may be implemented by various known methods using a cathode ray tube (CRT), a liquid crystal display (LCD), a light emitting diode (LED), a plasma display panel (PDP), an organic light emitting diode (OLED), and the like.

The input unit 290 may be implemented by various known methods using a keyboard, a foot switch, a foot pedal, and the like.

For example, a keyboard may be implemented as a hardware keyboard. The keyboard may include at least one among a switch, a key, a joystick, and a trackball, and may also be implemented as a software keyboard using a graphic user interface. In this case, the keyboard may be displayed on the display 280.

Meanwhile, in a case in which the display 280 is implemented as a touch screen type display, the display 280 may also perform a function of the input unit 290. That is, the main body 200 may receive various commends from the user via at least one of the display 280 and the input unit 290. As one embodiment, the display 291 illustrated in FIG. 1 may simultaneously perform a display function and an input function.

The display 280 and the input unit 290 may be defined as an input and output portion 270 because the input and output portion 270 receives or transmits information from or to the user.

FIG. 2 is a perspective view illustrating the ultrasonic probe according to one embodiment of the present disclosure.

Referring to FIG. 2, the ultrasonic probe 100 according to one embodiment of the present disclosure includes a case 110 in which an ultrasonic transceiver, which is a transducer module, is accommodated, and the connecting cable 93 configured to connect the transceiver and the main body 200 of the ultrasonic diagnostic apparatus 300.

A lens 140 is disposed at one end of the case 110 to which the connecting cable 93 is not connected. The transducer module disposed in the case 110 may irradiate an object with an ultrasonic wave via the lens 140.

When a user uses the ultrasonic probe 100, the connecting cable 93 may be severely bent or twisted at the other end of the case 110 of the ultrasonic probe 100. When the connecting cable 93 is severely bent or twisted, the connecting cable 93 may be disconnected or a jacket of the connecting cable 93 may be damaged. In order to prevent sudden bending or twisting of the connecting cable 93 at the other end of the case 110 of the ultrasonic probe 100, the ultrasonic probe 100 may include a strain reliever 150, which is provided to surround the connecting cable 93, formed at the other end of the case 110 connected to the connecting cable 93. That is, the strain reliever 150 is provided outside the other end of the case 110 to prevent damage to the connecting cable 93.

The strain reliever 150 may be formed of a soft material such that the connecting cable 93 is smoothly bent. Although the strain reliever 150 is formed of the soft material, the strain reliever 150 has a predetermined rigidity to prevent sudden bending of the cable. Accordingly, it is preferable for the strain reliever 150 to have the predetermined rigidity, and a structure in which the connecting cable 93 is easily bent in one direction or a structure in which the cable is foldable in multiple stages.

In the disclosed disclosure, a coated layer formed of an ultraviolet (UV)-sensitive material having a property of light discoloration may be formed on an entirety or part of an outer surface of the ultrasonic probe 100. More specifically, the coated layer may be formed of the UV-sensitive material provided to respond to light having a wavelength ranging from 100 to 300 nm and having the property of light discoloration.

As a coated layer S formed of the UV-sensitive material having the property of light discoloration is formed on at least one surface among the case 110, the lens 140, and the strain reliever 150 of the ultrasonic probe 100 according to the disclosed disclosure, visual information about UV sterilization can be acquired, and disinfection information can be visualized.

The coated layer S may include a coated layer coated with a pigment in which a powder type material having a property of light discoloration and at least one of an organic solvent and a water-soluble solution are mixed.

More specifically, the UV-sensitive material having the property of light discoloration may include at least one among a fulgide-based molecule, a diarylethene-based molecule, an azobenzene-based molecule, a spirooxazine-based molecule, and a spiropyran-based molecule.

In addition, the diarylethene-based molecule may include at least one selected from the group consisting of thiophene perfluoropentane, benzothiophene perfluoropentane, benzothiophene maleicanhydride, benzothiophene cyanoethene, a benzothiophene perfluoropentane derivative, a benzothiophene sulfone perfluoropetene derivative, a benzothiophene perfluoropetene polymer, and a benzothiophene sulfone perfluoropetene polymer. However, the above-described examples are examples of the UV-sensitive material having the property of light discoloration, and examples of the UV-sensitive material are not limited thereto.

Hereinafter, a principle of light discoloration of the coated layer provided as the UV-sensitive material according to the disclosed disclosure will be described for the sake of understanding.

The property of light discoloration is a phenomenon in which light absorption characteristics of molecules or crystals are reversibly changed by an action of light. Specifically, the property of light discoloration is a phenomenon in which a color is reversibly changed as a chemical bonding state of a single species changes to another isomer thereof.

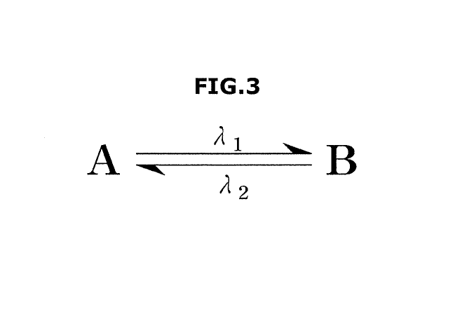

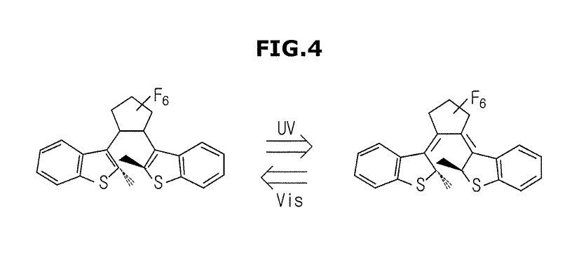

FIGS. 3 and 4 are views for describing the principle of light discoloration.

As illustrated in FIG. 3, molecules having the property of light discoloration may be reversibly isomerized by light having a specific wavelength because a structure of the molecules is changed and a degree of light absorption thereof is changed.

As illustrated in FIG. 4, as one example, a structure of 1,2-bis(2-methylbenzo[b]thiophene-3-yl) perfluorocyclopentene (BTF6) molecules in diarylethene is converted to have a closed form by UV light and discolored to be red, as illustrated in FIG. 4, and, conversely, when the molecules are irradiated with visible light, the structure of the molecules is converted to have an open form and a colorless state, and thus the degree of the light absorption is changed.

Such a property of light discoloration is caused by conjugation lengths in the molecules being modulated due to photoisomerization. Since electron transition is possible even at low energy in a visible light region when the molecules are exposed to UV light, the structure of the molecules is converted to have a closed form and the conjugation lengths are elongated, the molecules absorb the light and a color is expressed. Conversely, when the molecules are irradiated with visible light, the structure of the molecules is converted to have an open form, and the conjugate lengths are shortened so that absorption energy of the molecules is increased and the color disappears.

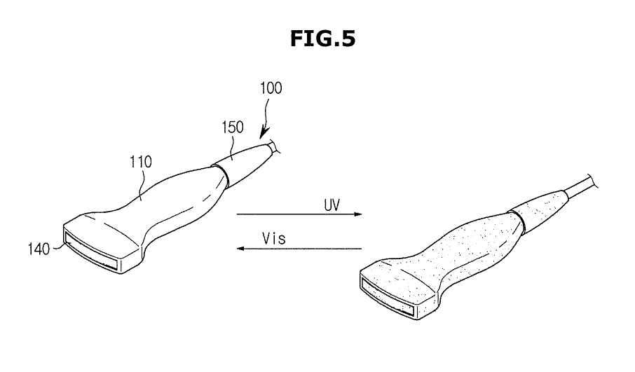

FIG. 5 is a view illustrating a discoloration process of the ultrasonic probe according to one embodiment of the present disclosure.

Referring to FIG. 5, according to one embodiment of the present disclosure, a surface of the ultrasonic probe to which the coated layer is applied is discolored during UV irradiation, and a color of the discolored surface of the ultrasonic probe is restored to its original color during visible light irradiation.

FIG. 5 shows a case in which the coated layer is applied to all of the case 110, the lens 140, and the strain reliever 150 of the ultrasonic probe. In this case, all of the surfaces of the case 110, the lens 140, and the strain reliever 150 of the probe may be discolored, and a user may be provided with disinfection information by visually checking the discoloration of the surfaces during UV irradiation.

Meanwhile, according to the embodiment, an additional indicator may be installed outside a UV disinfection apparatus such that a user easily receives the disinfection information. The indicator may display UV irradiation information over time, and a degree of sterilization may be displayed over time according to the embodiment.

The embodiment of the ultrasonic probe has been described above. In the ultrasonic probe according to the disclosed disclosure, whether UV disinfection of the ultrasonic probe is completed can be visually checked so that a user can intuitively check whether the ultrasonic probe is UV disinfected.

In addition, the user can intuitively check a degree of sterilization of the ultrasonic probe according to a location on the UV disinfection apparatus for disinfection of the ultrasonic probe. Accordingly, in a case in which a UV lamp is worn and intensity thereof is reduced through use so that sterilization power is weakened, the user can intuitively confirm the weakened sterilization power, and thus the ultrasonic probe can be more reliably disinfected.

As described above, the embodiments of the ultrasonic probe have been described. The technical spirit of the present disclosure is not limited to the above described embodiments, and it should be widely understood that the spirit includes modifications within a range easily conceived of by those skilled in art.

What is claimed is:

1. An ultrasonic probe comprising a coated layer formed on a surface thereof and formed of an ultraviolet (UV)-sensitive material having a property of light discoloration,
wherein the UV-sensitive material provided in the coated layer is discolored during UV irradiation.

2. The ultrasonic probe of claim 1, wherein the coated layer is coated with a pigment in which a powder type material having the property of light discoloration and at least one of an organic solvent and a water-soluble solution are mixed.

3. The ultrasonic probe of claim 1, wherein the coated layer is formed on at least a part of a surface of the ultrasonic probe.

4. The ultrasonic probe of claim 1, further comprising:
a case configured to accommodate a transducer module; and
a lens installed at one end of the case,
wherein the coated layer is formed on at least one of the case and the lens.

5. The ultrasonic probe of claim 4, further comprising a strain reliever provided at the other end of the case, wherein the coated layer is formed on the strain reliever.

6. The ultrasonic probe of claim 1, wherein the UV-sensitive material having the property of light discoloration includes a UV-sensitive material having the property of light discoloration including at least one selected from the group consisting of a fulgide-based molecule, a diarylethene-based molecule, an azobenzene-based molecule, a spirooxazine-based molecule, and a spiropyran-based molecule.

7. The ultrasonic probe of claim 6, wherein the diarylethene-based molecule includes at least one selected from the group consisting of thiophene perfluoropentane, benzothiophene perfluoropentane, benzothiophene maleicanhydride, benzothiophene cyanoethene, a benzothiophene perfluoropentane derivative, a benzothiophene sulfone perfluoropetene derivative, a benzothiophene perfluoropetene polymer, and a benzothiophene sulfone perfluoropetene polymer.
